(19) United States
(12) Patent Application Publication
(10) Pub. No.: US 2024/0035952 A1
(43) Pub. Date: Feb. 1, 2024

(54) BLOOD ANALYSER WITH IMAGE PLANE ANALYSIS AND RELATED METHODS

(71) Applicant: Radiometer Medical ApS, Brønshøj (DK)

(72) Inventor: Heine HANSEN, Brønshøj (DK)

(21) Appl. No.: 18/258,390

(22) PCT Filed: Dec. 22, 2021
(86) PCT No.: PCT/EP2021/087405

§ 371 Date: Jun. 20, 2023

(30) Foreign Application Priority Data
Dec. 22, 2020 (EP) 20216600.5

(51) Int. Cl. G01N 15/14; G01N 33/49

(52) U.S. Cl. CPC G01N 15/1463; G01N 33/49; G01N 2015/008

(57) ABSTRACT

A blood analyser and related methods, in particular a method of analysing a blood sample is disclosed. The blood analyser comprises a memory, an interface, and one or more processors. The blood analyser is configured to obtain image data of a prepared blood sample; select a first image associated with a first image plane of the prepared blood sample from the image data; characterize the first image, wherein the characterization of the first image comprises to determine a first set of cell regions belonging to the first image plane; and determine a first blood parameter based on the first set of cell regions.

The present disclosure relates to blood sample analysis and related tools, methods, and systems in particular for determining one or more blood parameters. Thus, a blood analyser and related methods, in particular a method of analysing a blood sample is provided.

### BACKGROUND

Today the analysis of a blood sample, such as determining a blood parameter, may be lengthy and require numerous steps, preparation, resources, and various advanced equipment. It may especially be lengthy and extensive to determine a blood count, such as a complete blood count.

### SUMMARY

Accordingly, there is a need for blood analysers and related methods, in particular methods of analysing a blood sample with improved blood sample analysis, speed, and accuracy.

A blood analyser is disclosed. The blood analyser comprises a memory, an interface, and one or more processors. The blood analyser is configured to obtain image data of a prepared blood sample; select a first image associated with a first image plane of the prepared blood sample from the image data; characterize the first image, wherein the characterization of the first image comprises to determine a first set of cell regions belonging to the first image plane; and determine a first blood parameter based on the first set of cell regions. Optionally, the image data comprises a stack of images where each image of the stack of images is associated with an image plane, wherein each image plane is associated with a different height along a z-axis of the prepared blood sample. Optionally, the cell regions are indicative of one or more platelets.

Further, a method of analysing a blood sample is disclosed, wherein the method comprises obtaining image data of a prepared blood sample; selecting a first image associated with a first image plane of the prepared blood sample from the image data; characterizing the first image, wherein the characterization of the first image comprises determining a first set of cell regions belonging to the first image plane; and determining a first blood parameter based on the first set of cell regions. The method may be performed using a blood analyser as disclosed herein.

Also disclosed is a system comprising a microscope, an image acquiring device, a blood sample cavity for accommodating a prepared blood sample, and a blood analyser, wherein the blood analyser is a blood analyser according to the present disclosure.

It is an advantage of the present disclosure that an improved blood sample analysis is provided.

It is an advantage of the present disclosure that it provides a more efficient and faster blood sample analysis.

For example, a more efficient, precise, robust, and faster image-based blood parameter determination may be achieved, e.g. cell classification, such as the determination of platelet concentration in a blood sample. Further, an improved cell classification with higher accuracy is provided, and, in particular improved, platelet analysis.

It is an advantage of the present disclosure that it may allow to analyse a blood sample being prepared with less chemicals, e.g. a blood sample being less diluted than prepared blood samples being analysed today. For example, it may be sufficient to dilute a blood sample with a reagent to a dilution ratio of 2:1 of reagent:blood compared to the dilution ratio of 10000:1 for a coulter counter for analysing a blood sample today. It is an advantage of the present disclosure that it may not be needed to analyse single cells through a narrow tube, such as flow cytometry or a coulter counter. Instead, larger blood volumes may be examined or analysed. It is therefore an advantage of the present disclosure that a more robust system and/or method may be provided, for example avoiding clotting, requiring less maintenance than conventional systems, and have lower costs. It is an advantage of the present disclosure that less equipment, time, cost, and/or steps are required to analyse a blood sample. For example, it may be possible to count cells, such as platelets, in one single image plane. It may therefore be possible to detect an anomaly in a blood sample in a faster and more efficient manner. Further, it may be easier to integrate the blood analysers, the systems, and/or the methods of the present disclosure into blood gas analysers.

### BRIEF DESCRIPTION OF THE DRAWINGS

The above and other features and advantages of the present invention will become readily apparent to those skilled in the art by the following detailed description of example embodiments thereof with reference to the attached drawings, in which:

FIG. 1 schematically illustrates an example system comprising a blood analyser according to the present disclosure, and

FIGS. 2A-C are flow diagrams of an example method according to the present disclosure.

### DETAILED DESCRIPTION

Various example embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

The figures are schematic and simplified for clarity, and they merely show details which aid understanding the disclosure, while other details have been left out. Throughout, the same reference numerals are used for identical or corresponding parts.

In the following, whenever referring to proximal side of an image plane, the referral is to the side closest to or the surface facing a camera or sensor (e.g. of a microscope), when an image is obtained/captured. Likewise, whenever referring to the distal side of an image plane, the referral is to the side furthest away from or the surface facing away from the camera or sensor, when an image is obtained/captured. In other words, the proximal side or surface is the side or surface closest to the camera or sensor, when an image is obtained/captured and the distal side is the opposite side or surface with respect to the image plane. In other words, the distal side may be the side or surface closest to the bottom of a container containing the prepared blood sample when an image is obtained/captured.

A blood analyser is disclosed. The blood analyser comprises a memory, an interface, and one or more processors. The blood analyser may comprise an electronic device such as a computer, e.g. a laptop computer or PC, a tablet computer, and/or a mobile phone, such as a smartphone. The blood analyser may for example be a point of care (POC) device. The blood analyser may for example be configured to be integrated with a blood gas analyser. The blood analyser may for example be a user device, such as a computer or a mobile phone, configured to perform an analysis of a blood sample, such as a prepared blood sample. The blood analyser may for example be part of the equipment in a laboratory.

In one or more example blood analysers, the blood analyser is a server device, such as acting as a server device. In other words, the blood analyser may be seen as implemented on a server device, such as the blood analyser may be configured to run and/or operate on a server device. The blood analyser acting as server device may be seen as a device configured to act as a server in communication with a client device, such as a computer, e.g. a laptop computer or PC, a tablet computer, and/or a mobile phone, such as a smartphone. For example, the blood analyser may be a remote server device configured to communicate with a client device. The blood analyser acting as server device may for example be configured to perform any one or more of: obtaining image data, selecting a first image, characterizing the first image, and determining a first blood parameter. The blood analyser acting as server device may for example be configured to output the first blood parameter to a client device.

The blood analyser is configured to obtain image data, also denoted ID, of a prepared blood sample. Image data may comprise one or more images, such as a plurality of images, e.g. a stack of images. The image data may comprise a plurality of images of the prepared blood sample obtained with a microscope and a camera, such as a CMOS image sensor camera. The image data may for example at least comprise ten images, at least twenty images, at least thirty images, at least forty images, at least fifty images, or at least a hundred images. The images of the image data may have an area depending on the area of the camera image sensor and the microscope magnification, e.g. A=A_im/M², where A is the area of the captured image, M is the magnification of the microscope, and A_im is the area that the camera image sensor may capture. The images of the image data may have a pixel size in the range of 0.1 μm to 5 μm, such as 0.5 μm, 1 μm, or 2 μm, depending on the resolution of the camera and/or the microscope. The image data may comprise a plurality of images of the prepared blood sample, where each image of the plurality of images is associated with an image plane of the prepared blood sample. The microscope and camera may obtain/acquire the plurality of images by stepping an optical focus plane along a z-axis, such as in a vertical direction of the prepared blood sample. The image data may therefore comprise a plurality of images being associated with images planes, where each image plane is separated by a distance Δz to the next obtained/acquired image plane and/or the previous obtained/acquired image plane. Δz may be the stepping incrementation for each obtained/acquired image. The image data may comprise a plurality of images of the prepared blood sample, where each image may be associated with an image plane being equidistant from the next obtained/acquired image plane and/or the previous obtained/acquired image plane. For example, the image data may comprise a 3D image stack, such as a stack of images where each image of the image stack is associated with an image plane having a different associated height along the z-axis of the prepared blood sample. In other words, each image plane may be associated with a unique height in the prepared blood sample contained in a container, e.g. the prepared blood sample contained in a cuvette. The distance between two image planes may be denoted inter-image distance. The distance between two image planes may vary, for example, depending on the type of cell of interest. The distance between two image planes may also, for example, vary depending on the numerical aperture, NA, and therefore also the depth of field, DoF, of the microscope which is used. This may be to achieve the best possible optical resolution. The distance between two image planes may for example be in the range of 1 μm to 10 μm, such as in the range of 2 μm to 8 μm, in the range of 3 μm to 6 μm, in the range of 1 μm to 8 μm, and/or in the range of 1 μm to 6 μm, e.g., when the cell of interest is a platelet, e.g. since platelets have a diameter in the range of 1 μm to 5 μm, such as in the range of 2 μm to 3 μm. For example, the distance between two image planes may for example be 3 μm, 3.5 μm, 4 μm, 4.5 μm, 5 μm, 5.5 μm, 6 μm, 7 μm, 8 μm, 9 μm, and/or 10 μm. The distance between two image planes may for example be 5.04 μm, e.g. when the cell of interest is a platelet, e.g. since platelets have a diameter in the range of 1 μm to 5 μm, such as in the range of 2 μm to 3 μm. The image data may comprise a plurality of images of a central portion of the blood sample (such as the prepared blood sample). In other words, the image data may comprise a plurality of obtained/acquired images of the prepared blood sample representing areas or volumes of the prepared blood sample being located away from the edges of the container in which the prepared blood sample is contained. An advantage of having images of the prepared blood sample representing areas or volumes of the prepared blood sample being located away from the edges of the container in which the prepared blood sample is contained may be to avoid seeing the edges of the glass of the container, such as dirt on the glass of the container. In one or more example blood analysers, the image data, such as one or more images of the image data, may be cropped. For example, an image taken with a resolution of 20 megapixels may be cropped by cropping 20% of the side length of the field of view, FOV. The cropped image may be of a central portion of the blood sample, such as a reduced part of the FOV. A central portion of the blood sample may have the best optical resolution and have the least optical aberration. An advantage of using cropped images may be that a larger number of images may be selected from the image data. For example, substantially all the images of the image data may be selected, such as at least 20 images, at least 30 images, or at least 40 images. By selecting more images, it may be possible to compensate for an inaccurate distance travel in a focus mechanism (such as a mechanical delta Z movement) of a microscope. A further advantage of using cropped images is that the computing resources for characterizing images, such as determining a set of cell regions, is reduced.

Each image of the image data ID may comprise a plurality of representations. The plurality of representations may comprise a plurality of particles, such as cells, e.g. white blood cells, WBCs, platelets, red blood cells, RBCs, clots of blood components, cell debris, and/or external particles, e.g. dust, precipitation, or residues from the container or the like. The plurality of representations may comprise a plurality of cells, e.g. mature cells such as Reticulocytes, Lymphocytes, and/or Monocytes, segmented and band-shaped Granulocytes: Neutrophil, Eosinophil, and/or Basophil, and immature cells such as Normoblasts, erythroblasts, proerythroblasts, Metamyelocytes, Myelocytes, Promyelocytes, Myeloblasts, Monoblasts, and/or Lymphoblasts.

The prepared blood sample may comprise a blood sample prepared with one or more reagents, chemicals, treatments, and/or processes. The prepared blood sample may for example comprise a blood sample which has been stained, such as chemically stained. The prepared blood sample may for example comprise a blood sample which has been hemolyzed, for example, wherein most of the red blood cells in the blood sample have been removed. The prepared blood sample may for example comprise a blood sample which has been positioned/fixed, such that substantially no cell movement occurs while obtaining/acquiring the images of the prepared blood sample. The prepared blood sample may be understood as a solution comprising blood and one or more reagents and/or chemicals. The prepared blood sample may be understood as a dissolution, e.g. a dissolved blood sample. The prepared blood sample may be placed/positioned in a container, such as a cuvette, while the image data, e.g. the plurality of images of the prepared blood sample, is obtained/acquired. The height that the image planes are associated with may be a height or distance, e.g. on the z-axis, with respect to the bottom of the container/cuvette or with respect to the camera/microscope, when the image was obtained/captured. The image planes that the plurality of images may extend in a two dimensional plane, e.g. a x-y-plane perpendicular with respect to the z-axis.

The blood analyser is configured to select an image, also denoted I_i, where i is the number of the selected image, associated with an image plane, also denoted IP_i, of the prepared blood sample from the image data ID. The blood analyser may be configured to select a first image, also denoted I_1, associated with a first image plane IP_1 of the prepared blood sample from the image data ID. In other words, to select an image I_i may comprise to select a first image I_1 associated with a first image plane IP_1 of the prepared blood sample from the image data ID. The first image I_1 may be selected from a plurality of images obtained from the image data ID. Optionally, the blood analyser may be configured to select a second image I_2, a third image I_3, a fourth image I_4, and/or a fifth image I_5. In one or more example blood analysers, the blood analyser may be configured to select more images, such as ten images, twenty images, or more. The images selected from the image data may be selected from a set of images, e.g. at least images each associated with an image plane of the prepared blood sample. In one or more example blood analysers, the blood analyser may be configured to select all, such as substantially all, e.g. more than 80% of, images of the stack of images. For example, substantially all the images of the image data may be selected, such as at least 20 images, at least 30 images, or at least 40 images. In one or more example blood analysers, the blood analyser may be configured to select more than 80% of the images of the stack of images.

In one or more example blood analysers, the first image plane is associated with a first height, also denoted H_1, in the prepared blood sample. The first height that the first image plane is associated with may be a height, e.g. on the z-axis, with respect to the bottom of the container/cuvette or with respect to the camera/microscope, when the first image was obtained/captured.

The blood analyser is configured to characterize the image I_i. In one or more example blood analysers, the characterization of the image I_i comprises to determine a set of cell regions, also denoted SCR_i belonging to the image plane IP_i. A cell region, also denoted CR_k, k=1, 2, . . . K, where K is the number of cell regions in the set of cell regions SCR_i, may be understood as a group of pixels in the image I_i representing one or more cells, a part of a cell, or an optical phenomena relating to a cell. In other words, the set of cell regions SCR_i comprises one or more cell regions, e.g. one or more group of pixels in the image I_i representing one or more cells, one or more parts of one or more cells, or optical phenomena relating to one or more cells. A cell region may preferably represent a single cell, a part of a single cell, or an optical phenomenon relating to a single cell. For example, an optical phenomenon of a bright area, e.g. bright spot, may occur in a proximal image plane PIP_i being on the proximal side, which is closest to the camera. This may occur when a mixture, such as the prepared blood sample, has a refractive index lower than a refractive index of a cell represented by the cell region. For example, an optical phenomenon of a dark area, e.g. dark spot, may occur in a distal image plane DIP_i being on the distal side, which is furthest from the camera. Optionally, when a mixture, such as the prepared blood sample, has a refractive index higher than a refractive index of a cell represented by the cell region, the optical phenomena of the bright area and the dark area may be inverted. In one or more example blood analysers, a bright spot detection may be performed and/or measured in a neighbouring image plane of the first image plane IP_1. For example, a bright spot detection may be performed and/or measured in an image plane at least one, two, three, five and/or ten image planes away from the first image plane IP_1. In other words, a bright spot detection may be performed and/or measured in the first distal image plane DIP_1 and/or the first proximal image plane PIP_1 being at least one, two, three, five and/or ten image planes away from the first image plane IP_1. For example, a bright spot detection may be performed and/or measured in the first distal image plane DIP_1 and/or the first proximal image plane PIP_1 being three image planes away from the first image plane IP_1. A bright spot may be detected, measured, and/or identified as a maximum positive contrast compared with the background intensity. For detecting a bright spot a bright spot criterion may be applied. The bright spot criterion may comprise a bright spot threshold, e.g., the bright spot threshold being of at least 15 count in intensity compared to the background. A background of an image may typically be of 200 count in intensity. For example, when a platelet cell is analysed, the platelet may be weak light absorbing in general. A lens effect (optical phenomenon/effect) may also occur when analysing a platelet which generates a bright area, such as a white dot, in the distal image plane DIP_i and/or proximal image plane PIP_i and may be observed in the distal cell region DCR_k_i and/or the proximal cell region PCR_k_i associated with the cell region CR_k_i. Other particles in the size range of platelets (1 μm to 5 μm) do not generate a lens effect. The platelets and other particles in the same size range can therefore be differentiated. For example, particles like reticulocyte nucleus fragments and reagent crystalline deposits may not produce a bright spot, and may thereby be differentiated from a platelet cell producing a bright spot. Bright spot detection may be used to sort, filter, and/or suppress unwanted particles from the prepared blood sample. The blood analyser may be configured to characterize the first image I_1. In one or more example blood analysers, the characterization of the first image I_1 comprises to determine a first set of cell regions SCR_1 belonging to the first image plane IP_1. In other words, to characterize the image I_i may comprise to characterize the first image I_1. In other words, to determine a set of cell regions SCR_i belonging to the image plane IP_i may comprise to determine a first set of cell regions SCR_1 belonging to the first image plane IP_1. In other words, to determine a set of cell regions SCR_i belonging to the image plane IP_i may comprise to determine a first set of cell regions SCR_1 being in focus in or associated with the first image plane IP_1. In other words, the first set of cell regions SCR_1 may comprise one or more cell regions, e.g. one or more groups of pixels in the first image I_1 representing one or more cells, one or more parts of one or more cells, or optical phenomena relating to one or more cells. Belonging to the image plane IP_i, such as belonging to the first image plane IP_1, may be understood as cell regions CR_k representing cells (e.g. the volume of the cell) being located mostly in the image plane IP_i, at the time when the image was obtained/captured, or cell regions being assigned to or in focus in an image plane. For example, belonging to the image plane IP_i may be understood as cell regions CR_k representing cells being located mostly in the volume around the image plane IP_i, such as centred around the image plane IP_i. For example, belonging to the image plane IP_i may be understood as cell regions CR_k representing cells being located in the volume around the image plane IP_i. For example, when the distance D between the image plane IP_i and the next neighbouring image plane is 5.04 μm, the belonging to the image plane IP_i may be understood as cell regions CR_k representing cells being located in the volume +2.02 μm and −2.02 μm around the image plane IP_i. Belonging to the image plane IP_i may be understood as cell regions CR_k representing cells being located in the volume between the image plane IP_i and the next neighbouring image plane, e.g. the next neighbouring distal image plane and/or the next neighbouring proximal image plane. In one or more example blood analysers, a set of cell regions may extend in more than one image plane. For example, when a cell region represents a cell larger than the distance between two image planes, the cell may extend to more than one image plane. In one or more example blood analysers, a set of cell regions may be assigned to an image plane, such as the first image plane IP_1. In one or more example blood analysers, the distance between two neighbouring image planes may be based on the size of a cell type of interest, e.g. to make sure that a cell belongs to an image plane. For example, a white blood cell having a diameter in the range e.g. of 5 μm to 10 μm may belong to two image planes.

The blood analyser is configured to determine a first blood parameter, also denoted BP_1, based on the set of cell regions SCR_i. In one or more example blood analysers, the blood analyser is configured to determine a first blood parameter BP_1 based on the first set of cell regions. To determine a first blood parameter BP_1 may comprise to determine a first number of cell regions in the first set of cell regions SCR_1, where the first blood parameter BP_1 is based on the first number of cell regions. The first blood parameter may comprise one or more cell counts, such as a white blood cell count and/or a platelet count. The first blood parameter may comprise one or more cell concentrations, such as a white blood cell concentration or a platelet concentration.

To determine a first blood parameter BP_1 may comprise to count the number, also denoted C_i, where i is the number of the selected image plane, of cell regions CR_i in one or more sets of cell regions SCR_i. To determine a first blood parameter BP_1 may comprise to count the number of cell regions CR_i in one or more image planes IP_i, such as the first image plane IP_1, the second image plane IP_2, the third image plane IP_3, the fourth image plane IP_4, and/or the fifth image plane IP_5. When the number of cell regions CR_i have been counted for more than one set of cell regions SCR_i, e.g. for more than one image plane IP_i, the result of the count of cell regions may be averaged based on the number of set of cell regions SCR_i. Belonging to the image plane IP_i, such as belonging to the first image plane IP_1, may be understood as belonging to a volume V located between the image plane IP_i and the next neighbouring distal image plane DIP_i and/or the next neighbouring proximal image plane PIP_i. A volume V between two image planes, may for example be in the range of 2 nL to 50 nL, such as in the range of 3 nL to 25 nL, e.g. 3.22 nL for a colour system (using an RGB colour camera) or 4.06 nL for a monochrome system (using a monochrome camera with monochrome light source and with no RGB filters). The volume V may be defined as V=D·A, where D is the distance from the image plane IP_i and the next neighbouring distal image plane DIP_i and/or the next neighbouring proximal image plane PIP_i, and A is the image sensor field of view, FOV, in the image plane IP_i. In one or more example blood analysers, D may be the distance between the windows of the container (such as distance between the windows of a cuvette). A may be defined as A=A_im/M², where A is the area of the captured image, M is the magnification of the microscope, and A_im is the area that the camera image sensor may capture. To determine a first blood parameter BP_1 may comprise to determine a cell concentration, also denoted c, of the cell of interest. The concentration of cells in the prepared blood sample may be defined as c=C_i/V. To determine a first blood parameter may comprise to determine a cell concentration c, where the cell concentration c may be compensated for dilution by one or more solutions/chemicals, such as reagents, and/or a fluid transport system. To determine a first blood parameter BP_1 may comprise to classify each cell region of the set of cell regions SCR_i. To determine a first blood parameter BP_1 may comprise to determine a deficiency in the prepared blood sample, such as a cell anomaly. In one or more example blood analysers, a quality control may be performed by analysing a test sample comprising a plurality of cell sized beads, such as platelet sized plastic beads, using the blood analyser according to the disclosure. This may allow to have a quality control of the blood parameter BP_i.

In one or more example blood analysers, to determine a first blood parameter BP_1 may comprise to compensate for larger cells than the cell of interest, such as compensate in the count of number of cell regions C_1 in the first set of cell region SCR_1. For example, when the cell of interest is a platelet, it may not be possible to identify/detect platelets in a region with a high concentration of WBC, since WBC are much larger than platelets, e.g. times larger. It may therefore be advantageous to compensate for the areas/volumes in the image I_i where large objects, such as WBC, are present. For example, a total area A_large of where the large objects are in the image I_i may be determined and compared to the total area of the image A_im. A fraction F=A_large/A_im may be determined. The compensated number of cell regions C_comp_1 may then be C_comp_1=C_1/(1−F).

In one or more example blood analysers, the blood analyser is configured to select, from the image data ID, a distal image, also denoted DI_i, where i is the number of the selected distal image, associated with a distal image plane, also denoted DIP_i on a distal side of the image plane IP_i. To select a distal image DI_i may comprise to select a first distal image DI_1 associated with a first distal image plane DIP_1, on a distal side of the first image plane IP_1. In other words, the distal image DI_i may comprise a first distal image DI_1. The distal side may be understood as the side or surface closest to the bottom of a container containing the prepared blood sample when the distal image is obtained/captured. In one or more example blood analysers, the blood analyser is configured to characterize the first distal image DI_1.

In one or more example blood analysers, the first distal image plane DIP_1 is associated with a first distal height DH_1 in the prepared blood sample. The first distal height DH_1 that the first distal image plane DIP_1 is associated with may be a distal height, e.g. on the z-axis, with respect to the bottom of the container/cuvette or with respect to the camera/microscope, when the first distal image was obtained/captured. The first distal height DH_1 is different from the first height H_1. In other words, the distance between the first image plane IP_1 and the first distal image plane DIP_1 may be denoted as Δz being the stepping incrementation for each obtained/acquired image when the first distal image plane DIP_1 is the image plane right after the first image plane IP_1 with respect to the camera, i.e. the direct distal neighbouring image plane with respect to the first image plane IP_1. In one or more example blood analysers, the distance between the first image plane IP_1 and the first distal image plane DIP_1 may be larger than Δz, e.g. 2·Δz, 3·Δz, 4·Δz, or 5·Δz, for example when the first distal image plane DIP_1 is an image plane after the first image plane IP_1 with respect to the camera but not the direct distal neighbouring image plane with respect to the first image plane IP_1, i.e. the first distal image plane DIP_1 being more than one image plane away from the first image plane IP_1.

In one or more example blood analysers, the determination of the set of cell regions SCR_i is based on the distal image DI_i. In other words, the determination of the first set of cell regions SCR_1 may be based on the first distal image DI_1.

In one or more example blood analysers, the blood analyser is configured to select, from the image data ID, a proximal image, also denoted PI_i, where i is the number of the selected proximal image, associated with a proximal image plane, also denoted PIP_i, on a proximal side of the image plane IP_i. To select a proximal image PI_i may comprise to select a first proximal image PI_1 associated with a first proximal image plane PIP_1, on a proximal side of the first image plane IP_1. In other words, the proximal image PI_i may comprise a first proximal image PI_1. The proximal side may be understood as the side or surface closest to or facing a camera or sensor when the proximal image is obtained/captured. In one or more example blood analysers, the blood analyser is configured to characterize the first proximal image PI_1.

In one or more example blood analysers, the first proximal image plane PIP_1 is associated with a first proximal height PH_1 in the prepared blood sample. The first proximal height PH_1 that the first proximal image plane PIP_1 is associated with may be a proximal height, e.g. on the z-axis, with respect to the bottom of the container/cuvette or with respect to the camera/microscope, when the first proximal image was obtained/captured. The first proximal height PH_1 is different from the first height H_1. In other words, the distance between the first image plane IP_1 and the first proximal image plane PIP_1 may be denoted as Δz being the stepping incrementation for each obtained/acquired image when the first proximal image plane PIP_1 is the image plane just prior to the first image plane IP_1 with respect to the camera, i.e. the direct proximal neighbouring image plane with respect to the first image plane IP_1. In one or more example blood analysers, the distance between the first image plane IP_1 and the first proximal image plane PIP_1 may be larger than Δz, e.g. 2·Δz, 3·Δz, 4·Δz, or 5·Δz, for example when the first proximal image plane PIP_1 is an image plane prior to the first image plane IP_1 with respect to the camera but not the direct proximal neighbouring image plane with respect to the first image plane IP_1, i.e. the first proximal image plane PIP_1 being more than one image plane away from the first image plane IP_1.

In one or more example blood analysers, the determination of the set of cell regions SCR_i is based on the proximal image PI_i. In other words, the determination of the first set of cell regions SCR_1 may be based on the first proximal image PI_1.

In one or more example blood analysers, a distal distance also denoted DD_i between the image plane IP_i and the distal image plane DIP_i, and a proximal distance also denoted PD_i between the image plane IP_i and the proximal image plane PIP_i are equal, such as equidistant planes. For example, a first distal distance also denoted DD_1 between the first image plane IP_1 and the first distal image plane DIP_1, and a first proximal distance also denoted PD_1 between the first image plane IP_1 and the first proximal image plane PIP_1 are equal. Equal may be seen as substantially equal. Substantially may refer to an amount that is within less than or equal to 10% of, within less than or equal to 5% of, within less than or equal to 1% of, within less than or equal to 0.1% of, and within less than or equal to 0.01% of the stated amount.

The distance between two image planes, such as the first distal distance also denoted DD_1 between the first image plane IP_1 and the first distal image plane DIP_1, and a first proximal distance also denoted PD_1 between the first image plane IP_1 and the first proximal image plane PIP_1 may be in the range from 1 μm to 10 μm, such as in the range of 2 μm to 8 μm, in the range of 3 μm to 6 μm, in the range of 1 μm to 8 μm, and/or in the range of 1 μm to 6 μm, e.g., when the cell of interest is a platelet, e.g. since platelets have a diameter in the range of 1 μm to 5 μm, such as in the range of 2 μm to 3 μm. For example, a first distal distance DD_1 between the first image plane IP_1 and the first distal image plane DIP_1, and a first proximal distance also denoted PD_1 between the first image plane IP_1 and the first proximal image plane PIP_1 may for example be 3 μm, 3.5 μm, 4 μm, 4.5 μm, 5 μm, 5.5 μm, 6 μm, 7 μm, 8 μm, 9 μm, and/or 10 μm, e.g. when the cell of interest is a platelet, e.g. since platelets have a diameter in the range of 1 μm to 5 μm. The distance between two image planes, such as the distal distance DD_i and/or the proximal distance PD_i may for example be 5.04 μm, e.g. when the cell of interest is a platelet, e.g. since platelets have a diameter in the range of 1 μm to 5 μm.

The distance between two image planes may hence be defined based on the size/diameter of the cell of interest. For example, the distance between two image planes may hence be defined as the maximum diameter and/or extension in one direction of the cell of interest. The distance between two image planes may also, for example, vary depending on the numerical aperture, NA, and therefore also the depth of field, DoF, of the microscope which is used.

In one or more example blood analysers, the characterization of the image I_i, such as the first image I_1, comprises to determine an initial candidate set of candidate cell regions, also denoted ICCR_i, where i is the number of the selected image, in the image I_i. To determine an initial candidate set of candidate cell regions ICCR_i in the image I_i may comprise to determine a first initial candidate set of candidate cell regions ICCR_1 in the first image I_1. The first initial candidate set of candidate cell regions ICCR_1 may comprise one or more candidate cell regions, e.g. one or more group of pixels in the first image I_1 being candidates to represent one or more cells, one or more parts of one or more cells, or optical phenomena relating to one or more cells. A candidate cell region, also denoted CCR_f, f=1, 2, . . . F, where F is the number of candidate cell regions in the initial candidate set of candidate cell regions ICCR_i, may be understood as a group of pixels in the image I_i representing a candidate cell, a part of a candidate cell, or an optical phenomena relating to a candidate cell.

In one or more example blood analysers, to determine the initial candidate set of candidate cell regions ICCR_i comprises to determine a background image, also denoted BG_i, where i is the number of the selected image, of the image I_i, and wherein the initial candidate set of candidate cell regions ICCR_i is based on the background image BG_i. To determine a background image BG_i of the image I_i may comprise to determine a first background image BG_1 of the first image I_1, and wherein the first initial candidate set of candidate cell regions ICCR_1 is based on the first background image BG_1. To determine the background image BG_i may comprise to determine a moving average window of the image I_i, e.g. to create a uniform image that follows image variations because of the illumination. The moving average window may for example have a side length in the range of 30 μm to 100 μm. The background image BG_i may also be denoted as a background intensity image. Prior to the determination of the background image BG_i, the blood analyser may be configured to determine/convert the image I_i, such as the first image I_1, to a greyscale image, e.g. from an RGB image format of the image I_i.

In one or more example blood analysers, to determine the initial candidate set of candidate cell regions comprises to determine a contrast image, also denoted CI_i, where i is the number of the selected image, based on the background image BG_i and the image I_i. To determine a contrast image CI_i based on the background image BG_i and the image I_i may comprise to determine a first contrast image CI_1 based on the first background image BG_1 and the first image I_1. In one or more example blood analysers, the initial candidate set of candidate cell regions ICCR_i is based on the contrast image CI_i. The first initial candidate set of candidate cell regions ICCR_1 may be based on the first contrast image CI_1. To determine the contrast image CI_i may comprise to subtract the image I_i from the background image BG_i, e.g. subtracting pixel by pixel the image I_i from the background image BG_i. In one or more example blood analysers, to determine the contrast image CI_i may comprise to subtract the background image BG_i from the image I_i, e.g. subtracting pixel by pixel the background image BG_i from the image I_i.

In one or more example blood analysers, to determine the initial candidate set of candidate cell regions ICCR_i comprises to determine a binary image also denoted BI_i, where i is the number of the selected image, based on the contrast image CI_i, and wherein the initial candidate set of candidate cell regions ICCR_i is based on the binary image BI_i. To determine a binary image BI_i based on the contrast image CI_i may comprise to determine a first binary image BI_1 based on the first contrast image CI_1, and wherein the first initial candidate set of candidate cell regions ICCR_1 is based on the first binary image BI_1. To determine a binary image BI_i based on the contrast image CI_i may comprise to apply a contrast criterion, such as thresholding the contrast image CI_i, e.g. the first contrast image CI_1, to generate the binary image BI_i, e.g. the first binary image BI_1. For example, to determine a binary image BI_i based on the contrast image CI_i may comprise to apply a binary mask threshold, e.g. for each pixel in the contrast image CI_i. The binary mask threshold may vary depending on the prepared blood sample, such as the sample type. For example, the binary mask threshold may be 0.09 (e.g. with 23 counts) for a potassium ethylenediaminetetraacetic acid, EDTA, sample, and 0.075 (e.g. with 19 counts) for an iloprost added to heparin, IH, sample. For example, the binary image BI_i may comprise mostly dark areas and the remaining being candidate cell regions as brighter areas, or vice-versa.

In one or more example blood analysers, to determine the initial candidate set of candidate cell regions ICCR_i comprises to identify connected regions, also denoted COR_i, where i is the number of the selected image, in the binary image BI_i. To identify connected regions COR_i in the binary image BI_i may comprise to identify first connected regions COR_1 in the first binary image BI_1. In one or more example blood analysers, the initial candidate set of candidate cell regions ICCR_i is based on the connected regions COR_i in the binary image BI_i. The first initial candidate set of candidate cell regions ICCR_1 may be based on the first connected regions COR_1 in the first binary image BI_1. To identify connected regions COR_i may comprise to identify connected pixels, such as connected 1's or 0's in the binary image BI_i. The initial candidate set of candidate cell regions ICCR_i may comprise a list of regions identified to be connected in the binary image BI_i. To identify connected regions COR_i in the binary image BI_i may comprise to identify connected components, such as clusters of pixels or regions of pixels.

In one or more example blood analysers, to determine the initial candidate set of candidate cell regions ICCR_i comprises to determine whether each respective connected region of the connected regions COR_i satisfies an area criterion, also denoted AC_m, m=1, 2, . . . M, where M is the number of the connected regions COR_i in the initial candidate set of candidate cell regions ICCR_i. In one or more example blood analysers, the blood analyser is configured to in accordance with the determination that the respective connected region of the connected regions COR_i satisfies the area criterion AC_i, to include the respective connected region satisfying the area criterion AC_i as a candidate cell region CCR_f in the initial candidate set of candidate cell regions ICCR_i. To determine the first initial candidate set of candidate cell regions ICCR_1 may comprise to determine whether each respective first connected region of the connected regions COR_1 satisfies an area criterion AC_m, and in accordance with the determination that the respective first connected region satisfies the area criterion AC_m, to include the respective first connected region satisfying the area criterion AC_m as a candidate cell region CCR_f in the first initial candidate set of candidate cell regions ICCR_1. The area criterion AC_m may comprise an area range criterion within certain limits depending on the type of cell of interest, e.g. to classify each candidate cell region CCR_f. For example, the area criterion AC_m may be used/applied to remove/sort away one or more of noise, small regions e.g. representing small particles that cannot be cells, cell regions representing cells that are not cells of interest, e.g. cell regions representing cells bigger and/or smaller than the cells of interest. For example, when the cell of interest is a platelet, the regions potentially representing WBCs and RBC may be sorted away and not identified as candidate cell regions. When it is not determined that the respective first connected region of the connected regions COR_1 satisfies the area criterion AC_m, the respective first connected region of the connected regions COR_1 may be discarded, e.g. sorted away and not included in the first initial candidate set of candidate cell regions ICCR_1.

In one or more example blood analysers, the area criterion AC_m comprises a threshold cell region area in the range of 1 μm² to 25 μm². For example, when the type cell of interest is a platelet, the area criterion AC_m may discard the connected regions COR_i corresponding to a circle having a diameter of less or equal to 1.5 μm and/or larger than 4.5 μm. In other words, the area criterion AC_m may comprise a threshold for the largest extraction in one direction of the connected region COR_i and/or a smallest extraction in one direction of the connected region COR_i. The area criterion AC_m may comprise a threshold of pixel region range, such as threshold for clusters of pixels.

In one or more example blood analysers, to determine the initial candidate set of candidate cell regions ICCR_i comprises to determine whether each respective cell region, such as each respective connected region of the connected regions COR_i, satisfies an area criterion and a contrast criterion. In other words, the determination of the initial candidate set of candidate cell regions ICCR_i may be based on a determination of whether each of the respective cell regions of the initial candidate set of candidate cell regions ICCR_i satisfies a combination of an area criterion and a contrast criterion. In one or more example blood analysers, the characterization of the image I_i comprises to determine whether each of the respective candidate cell regions CCR_f of the initial candidate set of candidate cell regions ICCR_i satisfy a shape criterion. A shape criterion may comprise a circularity criterion.

A circularity criterion may be seen as a criterion associated with a circularity of a cell region, such as a circularity of a candidate cell region CCR_f. For example, when a cell region, such as candidate cell region CCR_f, is elongated, the cell region may not be a platelet. A circularity criterion may comprise a circularity threshold and/or circularity range depending on the cell to be identified, classified, and/or characterized. A circularity criterion may be seen as a circularity test. A circularity parameter may be determined with the following formula: 4*Area (A)*Pi (π)/(divided by) Perimeter\hat{ }2 (P²). A circularity parameter may be in the range of 0 to 1, where 1 is the circularity of a perfect circular disk. For example, when a cell region satisfies a circularity criterion, such as the cell region is above the circularity threshold and/or when the cell region is in a circularity range, the cell region may be characterized as a platelet. A circularity threshold may for example be 0.8 when detecting, identifying, classifying, and/or characterizing platelets. In one or more example blood analysers, the characterization of the image I_i comprises to determine whether each of the respective candidate cell regions CCR_f of the initial candidate set of candidate cell regions ICCR_i satisfies a first criterion, also denoted FC_j j=1, 2, . . . J, where J is the number of the candidate cell regions CCR_f in the initial candidate set of candidate cell regions ICCR_i. In one or more example blood analysers, the blood analyser is configured to in accordance with the determination that a respective candidate cell region CCR_f of the initial candidate set of candidate cell regions ICCR_i respectively satisfies the first criterion FC_j, to include the respective candidate cell region CCR_f in a first candidate set of cell regions, also denoted FCCR_i, where i is the number of the selected image I_i. In one or more example blood analysers, the set of cell regions SCR_i is based on the first candidate set of cell regions FCCR_i.

In one or more example blood analysers, the characterization of the first image I_1 comprises to determine whether each of the respective candidate cell regions, also denoted CCR_f, of the first initial candidate set of candidate cell regions ICCR_1 satisfies a first criterion FC_j, and in accordance with the determination that a respective candidate cell region CCR_f of the first initial candidate set of candidate cell regions ICCR_1 respectively satisfies the first criterion FC_j, to include the respective candidate cell region CCR_f in a first candidate set of cell regions FCCR_1, and wherein the first set of cell regions SCR_1 is based on the first candidate set of cell regions FCCR_1. In one or more example blood analysers, the first criterion FC_j comprises a contrast criterion that each of the respective candidate cell regions CCR_f of the first initial candidate set of candidate cell regions ICCR_1 have to satisfy to be included in the first candidate set of cell regions FCCR_1. The first criterion FC_j may comprise an intensity contrast criterion, such as a cell region intensity contrast. For example, the first criterion FC_j for a cell region may be satisfied if a contrast parameter of the cell region in the image is larger than a distal contrast parameter of the same cell region in the distal image and larger than a proximal contrast parameter of the same cell region in the proximal image.

When it is not determined that each of the respective candidate cell regions CCR_f satisfies the first criterion FC_j, the respective candidate cell region CCR_f may be discarded, e.g. sorted away and not included in the first candidate set of cell regions FCCR_i.

In one or more example blood analysers, the first criterion FC_j is based on a distal contrast parameter, also denoted DCP_i, of the distal image DI_i and a proximal contrast parameter PCP_i of the proximal image PI_i. For example, the first criterion FC_j may be based on a first distal contrast parameter DCP_1 of the first distal image DI_1 and a first proximal contrast parameter PCP_1 of the first proximal image PI_1.

In one or more example blood analysers, to determine whether each of the respective candidate cell regions CCR_f of the first candidate set of cell regions FCCR_i satisfies a first criterion FC_j comprises to determine whether a contrast parameter, also denoted CP_i, of the contrast image CI_i is larger than the distal contrast parameter DCP_i, such as a distal contrast image DCI_i, and larger than the proximal contrast parameter PCP_i, such as a proximal contrast image DCI_i. For example, to determine whether each of the respective candidate cell regions CCR_f of the first candidate set of cell regions FCCR_i satisfies a first criterion FC_j comprises to determine whether a first contrast parameter CP_1 of the first contrast image CI_1 is larger than the first distal contrast parameter DCP_1 and larger than the first proximal contrast parameter PCP_1.

In one or more example blood analysers, when it is determined that the contrast parameter CP_i of the contrast image CI_i is larger than the distal contrast parameter DCP_i and larger than the proximal contrast parameter PCP_i, the blood analyser is configured to include the respective candidate cell region CCR_f in the first candidate set of cell regions FCCR_i.

The distal contrast parameter DCP_i may comprise a contrast parameter, such as contrast intensity or value, of the respective candidate cell region CCR_f in the distal image plane DIP_i, such as in the first distal image plane DIP_1. In other words, the distal contrast parameter DCP_i may comprise a contrast parameter of the respective candidate cell region CCR_f as seen from or in the distal image plane DIP_i. In other words, the distal contrast parameter DCP_i may comprise a distal contrast image also denoted DCI_i for the corresponding respective candidate cell regions CCR_f.

The proximal contrast parameter PCP_i may comprise a contrast parameter, such as contrast intensity or value, of the respective candidate cell region CCR_f in the proximal image plane PIP_i, such as in the first proximal image plane PIP_1. In other words, the proximal contrast parameter PCP_i may comprise a contrast parameter of the respective candidate cell region CCR_f as seen from or in the proximal image plane PIP_i. In other words, the proximal contrast parameter PCP_i may comprise a proximal contrast image also denoted PCI_i for the corresponding respective candidate cell regions CCR_f.

The contrast parameter CP_i may comprise a contrast parameter, such as contrast intensity or value, of the respective candidate cell region CCR_f in the image plane IP_i, such as in the first image plane IP_1. In other words, the contrast parameter CP_i may comprise a contrast parameter of the respective candidate cell region CCR_f as seen from or in the image plane IP_i. In other words, the contrast parameter CP_i may comprise or be comprised in the contrast image CI_i for the corresponding respective candidate cell regions CCR_f. The contrast parameter CP_i may for example comprise a maximum contrast value of the candidate cell regions CCR_f, a percentile contrast value of the candidate cell regions CCR_f, a mean contrast value of the candidate cell regions CCR_f, and/or a standard deviation contrast value of the candidate cell regions CCR_f.

To determine whether the contrast parameter CP_i, of the contrast image CI_i is larger than the distal contrast parameter DCP_i and larger than the proximal contrast parameter PCP_i may comprise to determine whether the intensity contrast of the respective candidate cell region CCR_f in the image plane IP_i, such as in the contrast image CI_i, is larger than the intensity contrast of the respective candidate cell region CCR_f in the distal image plane DIP_i, such as in the distal contrast image DCI_i, and larger than the intensity contrast of the respective candidate cell region CCR_f in the proximal image plane PIP_i, such as in the proximal contrast image PCI_i.

In other words, when it is determined that the contrast parameter CP_i of the contrast image CI_i is larger than the distal contrast parameter DCP_i and larger than the proximal contrast parameter PCP_i, it may be an indication that the respective candidate cell region CCR_f belongs to the image plane IP_i and not to the distal image plane DIP_i or the proximal image plane PIP_i. Further, when it is determined that the contrast parameter CP_i of the contrast image CI_i is larger than the distal contrast parameter DCP_i and larger than the proximal contrast parameter PCP_i, it may be an indication that the respective candidate cell region CCR_f are more in focus in the image plane IP_i than in the distal image plane DIP_i and more in focus than in the proximal image plane PIP_i.

In one or more example blood analysers, the characterization of the image I_i comprises to determine whether each of the respective candidate cell regions, also denoted CCR_fi, fi=1, 2, . . . FI, where FI is the number of candidate cell regions in the first candidate set of candidate cell regions FCCR_i, of the first candidate set of candidate cell regions FCCR_i satisfies a second criterion, also denoted SC_n n=1, 2, . . . N, where N is the number of the candidate cell regions CCR_fi in the first candidate set of candidate cell regions FCCR_i. In one or more example blood analysers, the blood analyser is configured to in accordance with the determination that a respective candidate cell region CCR_fi of the first candidate set of candidate cell regions FCCR_i respectively satisfies the second criterion SC_n, to include the respective candidate cell region CCR_fi in a second candidate set of cell regions, also denoted SCCR_i, where i is the number of the selected image I_i. In one or more example blood analysers, the set of cell regions SCR_i is based on the second candidate set of cell regions SCCR_i.

In one or more example blood analysers, the characterization of the first image I_1 comprises to determine whether each of the respective candidate cell regions CCR_fi of the first candidate set of candidate cell regions FCCR_1 satisfies a second criterion SC_n, and in accordance with the determination that a respective candidate cell region CCR_fi of the first candidate set of candidate cell regions FCCR_1 respectively satisfies the second criterion SC_n, to include the respective candidate cell region CCR_fi in a second candidate set of cell regions SCCR_1, and wherein the first set of cell regions SCR_1 is based on the second candidate set of cell regions SCCR_1. In one or more example blood analysers, the second criterion SC_n comprises a contrast threshold criterion that each of the respective candidate cell regions CCR_fi of the first candidate set of candidate cell regions FCCR_1 have to satisfy to be included in the second candidate set of cell regions SCCR_i. The second criterion SC_n may comprise an intensity contrast threshold criterion, such as a cell region intensity contrast threshold. The second criterion SC_n may comprise that the respective candidate cell regions CCR_fi of the first candidate set of candidate cell regions FCCR_1 have to satisfy that the intensity contrast has to be equal or above 35% (of the maximum intensity contrast) of the 90% fractile (such as percentile) of the respective candidate cell regions CCR_fi of the first candidate set of candidate cell regions FCCR_1. Applying the second criterion SC_n may act as a filter rule to discard or suppress false positives in the first candidate set of cell regions FCCR_i.

When it is not determined that each of the respective candidate cell regions CCR_fi satisfies the second criterion SC_n, the respective candidate cell region CCR_fi may be discarded, e.g. sorted away and not included in the second candidate set of cell regions SCCR_i.

In one or more example blood analysers, the cell regions CR_k are indicative of, such as representing, one or more platelets, e.g. in the prepared blood sample.

In one or more example blood analysers, the blood analyser is configured to select a second image I_2 associated with a second image plane IP_2 of the prepared blood sample from the image data ID. The description of the selection of the first image I_1 may also apply to the selection of the second image I_2.

In one or more example blood analysers, the blood analyser is configured to characterize the second image I_2, wherein the characterization of the second image I_2 comprises to determine a second set of cell regions SCR_2 belonging to the second image plane IP_2. The description of the characterization of the first image I_1 may also apply to the characterization of the second image I_2, and the description of determination of the first set of cell regions SCR_1 belonging to the first image plane IP_1 may also apply to the determination of the second set of cell regions SCR_2 belonging to the second image plane IP_2.

In one or more example blood analysers, to determine a first blood parameter BP_1 is based on the second set of cell regions SCR_2. The description of determination of the first blood parameter BP_1 based on the first set of cell regions SCR_1 may also apply to the determination of the determination of the first blood parameter BP_1 based on the second set of cell regions SCR_2. To determine the first blood parameter BP_1 based on the second set of cell regions SCR_2 may comprise to determine the first blood parameter BP_1 based on the first set of cell regions SCR_2 and the second set of cell regions SCR_2.

In one or more example blood analysers, to determine a first blood parameter BP_1 based on the first set of cell regions SCR_1 and the second set of cell regions SCR_2 comprises to determine a first number of cell regions, also denoted C_1, in the first set of cell regions SCR_1 and a second number of cell regions C_2 in the second set of cell regions SCR_2, and wherein the first blood parameter BP_1 is based on the first number C_1 and the second number C_2. In one or more example blood analysers, to determine a first blood parameter BP_1 may be based on a third set of cell regions SCR_3 belonging to a third image plane IP_3 in a third image I_3, a fourth set of cell regions SCR_4 belonging to a fourth image plane IP_4 in a fourth image I_4, and/or a fifth set of cell regions SCR_5 belonging to a fifth image plane IP_5 in a fifth image I_5. In one or more example blood analysers, to determine a first blood parameter BP_1 may be based on further sets of cell regions SCR_i belonging to further image planes IP_i.

The first blood parameter may therefore be based on a third number C_3, a fourth number C_4, and/or a fifth number C_5.

When the first blood parameter BP_1 is based on more than one set of cell regions SCR_i, such as two, three, four, or five set of cell regions SCR_i, to determine the first blood parameter BP_1 may comprise to apply a third criterion, also denoted TC, to the two or more set of cell regions SCR_i. The third criterion TC may comprise an outlier criterion, such as a Dixon criterion. Applying the third criterion TC may comprise to determine one or more outliers, such as a single outlier (Dixon test), and discard or suppress the one or more outlier when determining the first blood parameter.

It is to be understood that a description of a feature in relation to method(s) is also applicable to the corresponding feature in blood analyser and/or system and vice-versa.

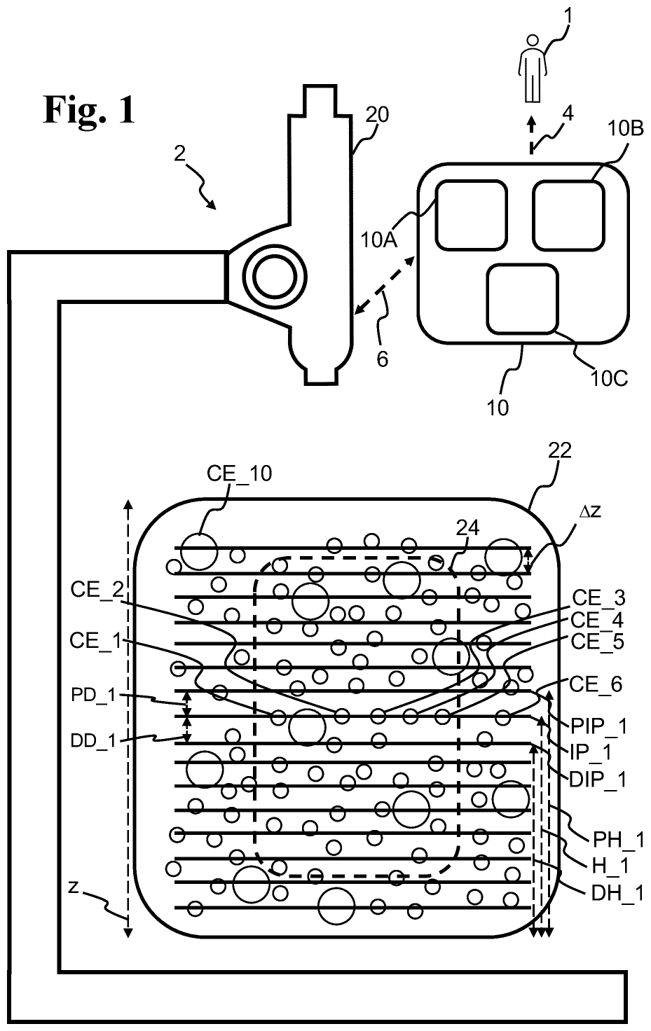

FIG. 1 schematically illustrates an example system 2, comprising a microscope 20, an image acquiring device (not shown, e.g. implemented/integrated with the microscope), a prepared blood sample in a container 22 (e.g., cuvette, cavity), and a blood analyser 10. The blood analyser 10 is a blood analyser according to the disclosure. The blood analyser 10 comprises a memory 10A, an interface 10B, and one or more processors, such as a processor 10C. The blood analyser 10 is configured to obtain 6 image data ID of a prepared blood sample, such as via the interface 10B from the image acquiring device. Optionally, the blood analyser 10 may be configured to obtain the image data from a network such as a global network, e.g. the internet or a telecommunications network. For example, the blood analyser 10 may be configured to obtain the image data from a server device (not shown), via the network. The prepared blood sample may be placed/positioned in a container 22, such as a cuvette, while the image data ID, e.g. the plurality of images of the prepared blood sample, is obtained/acquired, such as the first image I_1. The height that the image planes are associated with may be a height, e.g. on the z-axis, with respect to the bottom of the container 22 when the image was obtained/captured. The first image plane IP_1 may be associated with a first height H_1 in the prepared blood sample. The image planes that the plurality of images may extend in a two dimensional plane, e.g. a x-y-plane with respect to the z-axis. The image data ID may therefore comprise a plurality of images being associated with images planes, where each image plane is separated by a distance Δz to the next obtained/acquired image plane and/or the previous obtained/acquired image plane. Δz may be the stepping incrementation for each obtained/acquired image. In the example of FIG. 1, sixteen image planes are represented including the first image plane IP_1, the first distal image plane DIP_1, and the first proximal image pane PIP_1. The number of images and image planes may be increased to comprise for example at least thirty, at least forty, or at least a hundred. The image data ID may comprise a plurality of images of the prepared blood sample, where each image may be associated with an image plane being equidistant from the next obtained/acquired image plane and/or the previous obtained/acquired image plane. In other words, the image data ID may comprise a 3D image stack, such as a stack of images where each image of the image stack is associated with an image plane having a different associated height along the z-axis of the prepared blood sample. The image data ID may comprise a plurality of images of a central portion 24 of the blood sample. In other words, the image data ID may comprise a plurality of obtained/acquired images of the prepared blood sample representing areas or volumes of the prepared blood sample being located away from the edges of the container 22 in which the prepared blood sample is contained. Alternatively or additionally, the image data ID may comprise a plurality of obtained/acquired images of the prepared blood sample representing areas or volumes of the whole container 22, such as the full width and/or height of the container 22, e.g. including the windows of the container 22. The blood sample may comprise a plurality of cells, such as a first cell CE_1, a second cell CE_2, a third cell CE_3, a fourth cell CE_4, a fifth cell CE_5, and a sixth cell CE_6. For illustrative purposes the container 22 and the cells CE_1-CE_6 have been enlarged and are therefore not to scale. In the example shown in FIG. 1, the cells CE_1-CE_6 represent platelets. The larger cells, such as cell CE_10, may for example be white blood cells, WBC.

Each image of the image data ID may comprise a plurality of representations. The plurality of representations may comprise a plurality of particles, such as cells, e.g. white blood cells, WBCs, platelets, red blood cells, RBCs, and/or external particles, e.g. dust or residues from the container or the like.

The blood analyser 10 is configured to select a first image I_1, such as using the processor 10C, associated with a first image plane IP_1 of the prepared blood sample from the image data ID.

The blood analyser 10 is configured to characterize the first image I_1, wherein the characterization of the first image I_1 comprises to determine a first set of cell regions SCR_1 belonging to the first image plane IP_1. The first set of cell regions SCR_1 may be representative of the cells CE_1-CE_6.

The blood analyser 10 is configured to determine a first blood parameter BP_1 based on the first set of cell regions.

In one or more example systems and/or blood analysers, the blood analyser 10 is configured to select, from the image data ID, a first distal image DI_1 associated with a first distal image plane DIP_1 on a distal side of the first image plane IP_1, and wherein the determination of the first set of cell regions SCR_1 is based on the first distal image DI_1.

In one or more example systems and/or blood analysers, the blood analyser 10 is configured to select, from the image data ID, a first proximal image PI_1 associated with a first proximal image plane PIP_1 on a proximal side of the first image plane IP_1, and wherein the determination of the first set of cell regions SCR_1 is based on the first proximal image PI_1.

In one or more example systems and/or blood analysers, the first distal image plane DIP_1 is associated with a first distal height DH_1 in the prepared blood sample, the first distal height DH_1 being different from the first height H_1.

In one or more example systems and/or blood analysers, the first proximal image plane PIP_1 is associated with a first proximal height PH_1 in the prepared blood sample, the first proximal height PH_1 being different from the first height H_1.

A first distal distance also denoted DD_1 between the first image plane IP_1 and the first distal image plane DIP_1, and a first proximal distance also denoted PD_1 between the first image plane IP_1 and the first proximal image plane PIP_1 are in the range from 2.5 μm to 75 μm. A first distal distance also denoted DD_1 between the first image plane IP_1 and the first distal image plane DIP_1, and a first proximal distance also denoted PD_1 between the first image plane IP_1 and the first proximal image plane PIP_1 may be in the range from 1 μm to 10 μm, such as in the range of 2 μm to 8 μm, in the range of 3 μm to 6 μm, in the range of 1 μm to 8 μm, and/or in the range of 1 μm to 6 μm, e.g., when the cell of interest is platelets, e.g. since platelets have a diameter in the range of 1 μm to 5 μm, such as in the range of 2 μm to 3 μm. For example, a first distal distance DD_1 between the first image plane IP_1 and the first distal image plane DIP_1, and a first proximal distance also denoted PD_1 between the first image plane IP_1 and the first proximal image plane PIP_1 may for example be 3 μm, 3.5 μm, 4 μm, 4.5 μm, 5 μm, 5.5 μm, 6 μm, 7 μm, 8 μm, 9 μm, and/or 10 μm. In FIG. 1, the first distal distance DD_1 is larger than the first proximal distance PD_1.

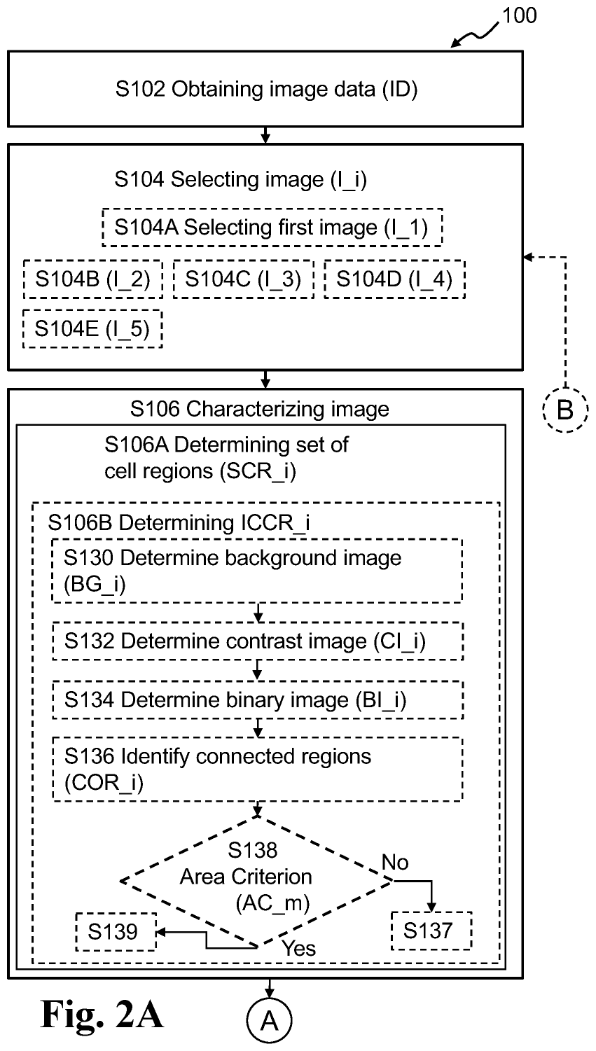

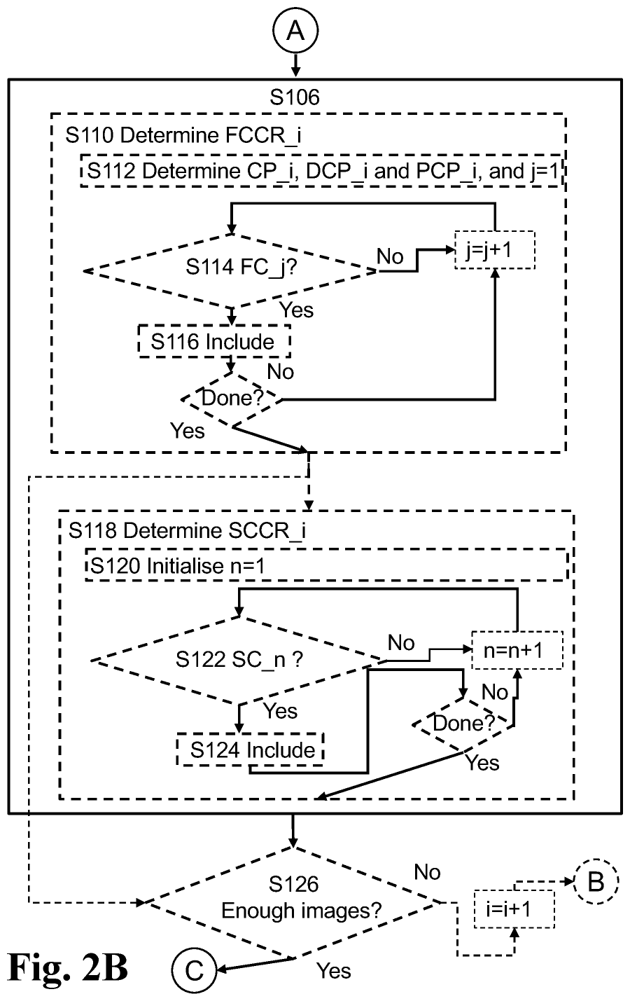

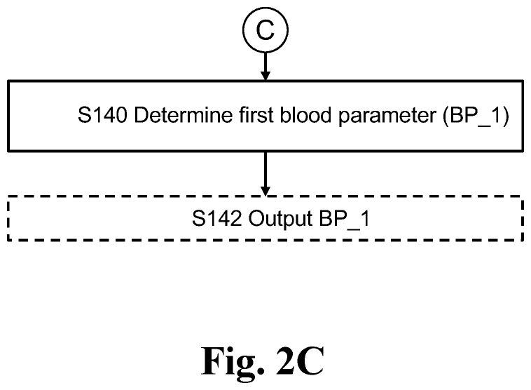

The blood analyser 10 may be configured to perform any of the methods disclosed in FIGS. 2A, 2B, 2C.

The blood analyser 10, such as the processor 100, is optionally configured to perform any of the operations disclosed in FIGS. 2A-2C (such as any one or more of S104A, S104B, S104C, S104D, S104E, S106B, S130, S132, S134, S136, S137, S138, S110, S112, S114, S116, S118, S120, S122, S124, S126, S142). The operations of the blood analyser may be embodied in the form of executable logic routines (for example, lines of code, software programs, etc.) that are stored on a non-transitory computer readable medium (for example, memory 10A) and are executed by the processor 10C).

Furthermore, the operations of the blood analyser 10 may be considered a method that the blood analyser 10 is configured to carry out. Also, while the described functions and operations may be implemented in software, such functionality may as well be carried out via dedicated hardware or firmware, or some combination of hardware, firmware and/or software.

FIGS. 2A, 2B, 2C show a flow diagram of an example method. A method 100 of analysing a blood sample, such as a prepared blood sample, is illustrated, the method 100 comprising obtaining S102 image data, also denoted ID, of a prepared blood sample.

The method 100 comprises selecting S104 an image, also denoted I_i, associated with an image plane, also denoted IP_i, of the prepared blood sample from the image data ID. The method may comprise selecting S104A a first image, also denoted I_1, associated with a first image plane IP_1 of the prepared blood sample from the image data ID. In other words, selecting S104 an image I_i may comprise selecting S104A a first image I_1 associated with a first image plane IP_1 of the prepared blood sample from the image data ID. The first image I_1 may be selected from a plurality of images obtained from the image data ID. Optionally, the method comprises selecting S104B a second image I_2, selecting S104C a third image I_3, selecting S104D a fourth image I_4, and/or selecting S104E a fifth image I_5.

The method 100 comprises characterizing S106 the image I_i. In one or more example methods, characterizing S106 the image I_i, such as the first image I_1, comprises determining a set of cell regions SCR_i, such as the first set of cell regions SCR_1, belonging to the image plane IP_i, such as the first image plane IP_1.

In one or more example methods, determining S106A a set of cell regions SCR_i comprises determining S106B an initial candidate set of candidate cell regions ICCR_i in the image I_i.

In one or more example methods, determining S106B the initial candidate set of candidate cell regions ICCR_i comprises determining S130 a background image BG_i of the image I_i, and wherein the initial candidate set of candidate cell regions ICCR_i is based on the background image BG_i.

In one or more example methods, determining S106B the initial candidate set of candidate cell regions ICCR_i comprises determining S132 a contrast image CI_i based on the background image BG_i and the image I_i, and wherein the initial candidate set of candidate cell regions ICCR_i is based on the contrast image CI_i.

In one or more example methods, determining S106B the initial candidate set of candidate cell regions ICCR_i comprises determining S134 a binary image BI_i based on the contrast image CI_i, and wherein the initial candidate set of candidate cell regions ICCR_i is based on the binary image BI_i.

In one or more example methods, determining S106B the initial candidate set of candidate cell regions ICCR_i comprises identifying S136 connected regions COR_i in the binary image BI_i, and wherein the initial candidate set of candidate cell regions ICCR_i is based on the connected regions COR_i in the binary image BI_i.

In one or more example methods, determining S106B the initial candidate set of candidate cell regions ICCR_i comprises determining S138 whether each respective connected region of the connected regions COR_i satisfies an area criterion AC_m. In one or more example methods, in accordance with the determination that the respective connected region satisfies the area criterion AC_m, including S139 the respective connected region satisfying the area criterion AC_m as a candidate cell region CCR_f in the initial candidate set of candidate cell regions ICCR_i. In one or more example methods, when it is not determined that the respective connected region of the connected regions COR_i satisfies the area criterion AC_m, the method comprises discarding S137 the respective connected region.

In one or more example methods, characterizing S106 the image I_i comprises determining S110 a first candidate set of cell regions FCCR_i.

In one or more example methods, characterizing S106 the image I_i comprises determining S114 whether each of the respective candidate cell regions CCR_f of the initial candidate set of candidate cell regions ICCR_i satisfies a first criterion FC_j, j=1, 2, . . . J, where J is the number of the candidate cell regions CCR_f in the initial candidate set of candidate cell regions ICCR_i.

In one or more example methods, in accordance with the determination that a respective candidate cell region CCR_f of the initial candidate set of candidate cell regions ICCR_i respectively satisfies the first criterion FC_j, the method comprises including S116 the respective candidate cell region in a first candidate set of cell regions FCCR_i, and wherein the set of cell regions SCR_i is based on the first candidate set of candidate cell regions FCCR_i.

In one or more example methods, when it is not determined that each of the respective candidate cell regions CCR_f satisfies the first criterion FC_j, the method comprises discarding the respective candidate cell region CCR_f and incrementing index j=j+1, and determining whether the next respective candidate cell region CCR_f of the initial candidate set of candidate cell regions ICCR_i satisfies a first criterion FC_j.

In one or more example methods, determining S110 a first candidate set of cell regions FCCR_i comprises determining S112 a contrast parameter CP_i of the image I_i, such as first contrast parameter CP_1 of the first image I_1. In one or more example methods, determining S110 a first candidate set of cell regions FCCR_i comprises determining S112 a distal contrast parameter DCP_i of the distal image DI_i, such as first distal contrast parameter DCP_1 of the first distal image I_1. In one or more example methods, determining S110 a first candidate set of cell regions FCCR_i comprises determining S112 a proximal contrast parameter PCP_i of the proximal image PI_i, such as first proximal contrast parameter PCP_1 of the first proximal image PI_1.

In one or more example methods, determining S110 a first candidate set of cell regions FCCR_i comprises initialising S112 index j=1 to start determining S114 whether each of the respective candidate cell regions CCR_f satisfies the first criterion FC_j.

In one or more example methods, the method 100 comprises determining whether all or substantially all the respective candidate cell region CCR_f of the initial candidate set of candidate cell regions ICCR_i have been checked, “Done?”. When it is not determined that all the respective candidate cell region CCR_f of the initial candidate set of candidate cell regions ICCR_i have been checked, the method comprises incrementing index j=j+1, and determining whether the next respective candidate cell region CCR_f of the initial candidate set of candidate cell regions ICCR_i satisfies a first criterion FC_j. When it is determined that all the respective candidate cell region CCR_f of the initial candidate set of candidate cell regions ICCR_i have been checked, the method 100 comprises determining S118 a second candidate set of candidate cell regions SCCR_i or determining S126 whether enough images I_i have been selected. When it is determined that enough images I_i have been selected, the method 100 comprises proceeding C to determining S140 a first blood parameter BP_1. When it is not determined that enough images I_i have been selected, the method 100 comprises reiterating B to selecting S104 a next image I_i.

In one or more example methods, characterizing S106 the image I_i comprises determining S118 a second candidate set of cell regions SCCR_i.

In one or more example methods, characterizing S106 the image I_i comprises determining S122 whether each of the respective candidate cell regions CCR_fi of the first candidate set of candidate cell regions FCCR_i satisfies a second criterion SC_n, n=1, 2, . . . N, where N is the number of the candidate cell regions CCR_fi in the first candidate set of candidate cell regions FCCR_i.

In one or more example methods, in accordance with the determination that a respective candidate cell region CCR_fi of the first candidate set of candidate cell regions FCCR_i respectively satisfies the second criterion SC_n, the method 100 comprises including S124 the respective candidate cell region in a second candidate set of cell regions SCCR_i, and wherein the set of cell regions SCR_i is based on the second candidate set of candidate cell regions SCCR_i.

In one or more example methods, when it is not determined that each of the respective candidate cell regions CCR_fi satisfies the second criterion SC_j, the method 100 comprises discarding the respective candidate cell region CCR_fi and incrementing index n=n+1, and determining whether the next respective candidate cell region CCR_fi of the first candidate set of candidate cell regions FCCR_i satisfies a second criterion SC_n.

In one or more example methods, determining S118 a second candidate set of cell regions SCCR_i comprises initialising S120 index n=1 for starting to determine S122 whether each of the respective candidate cell regions CCR_fi satisfies the second criterion SC_n.

In one or more example methods, the method 100 comprises determining whether all the respective candidate cell region CCR_fi of the first candidate set of candidate cell regions FCCR_i have been checked, “Done?”. When it is not determined that all the respective candidate cell region CCR_fi of the first candidate set of candidate cell regions FCCR_i have been checked, the method 100 comprises incrementing index n=n+1, and determining whether the next respective candidate cell region CCR_fi of the first candidate set of candidate cell regions FCCR_i satisfies a second criterion SC_n. When it is determined that all the respective candidate cell region CCR_fi of the first candidate set of candidate cell regions FCCR_i have been checked, the method 100 comprises determining S126 whether enough images I_i have been selected. When it is determined that enough images I_i have been selected, the method 100 comprises proceeding C to determining S140 a first blood parameter BP_1. When it is not determined that enough images I_i have been selected, the method 100 comprises reiterating B to selecting S104 a next image I_i.

The method 100 comprises determining S140 a first blood parameter BP_1 based on the set of cell regions SCR_i, such as the first set of cell regions SCR_1.

In one or more example methods, the method 100 comprises outputting S142 the first blood parameter BP_1, e.g. to a user of the blood analyser via an interface 10B of the blood analyser and/or to a server device.

In a first aspect of the invention, the blood analyser disclosed in present disclosure is configured to analyze biological fluids, such as, e.g., human, animal, mammalian blood, and/or cell cultures. Moreover, in said further aspect the blood analyser is substituted by and/or comprises a biological fluid analyser, such as, e.g., a blood analyser and/or a cell culture analyser.

In the first aspect, any disclosed blood sample may be substituted by and/or comprise a biological fluid sample, such, e.g., as a human blood sample, an animal blood sample, a mammalian blood sample, and/or a cell culture sample.

In the first aspect, any disclosed prepared blood sample may be substituted by and/or comprise a prepared biological fluid sample, such, e.g., as a prepared human blood sample, a prepared animal blood sample, a prepared mammalian blood sample, and/or a prepared cell culture sample.

In the first aspect, any disclosed blood parameter may be substituted by and/or comprise a biological fluid parameter, such as human blood parameter, an animal blood parameter, a mammalian blood parameter, and/or a cell culture parameter.

In some embodiments of the first aspect, the cell culture comprises a culture of cells derived from multicellular eukaryotes, such as, e.g., mammalian cells, animal cells, and/or human cells. In some embodiments, the cell culture comprises a culture of cells grown from plant tissue culture, fungal culture, and/or microbiological culture (of microbes).

In the first aspect, a cell may therefore be a mammalian cell, an animal cell, a human cell, a plant tissue cultured cell, a fungal cultured cell, or a microbiologically cultured cell.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Memory may be one or more of a buffer, a flash memory, a hard drive, a removable media, a volatile memory, a non-volatile memory, a random access memory (RAM), or other suitable device. In a typical arrangement, memory may include a non-volatile memory for long term data storage and a volatile memory that functions as system memory for the processor. Memory may exchange data with processor over a data bus. Memory may be considered a non-transitory computer readable medium.

Memory may be configured to store information (such as information indicative of the one or more audio signals, the one or more sentiment metrics, the one or more appearance metrics, the speaker representations, the sentiment metric data, and/or the appearance metric data) in a part of the memory.

Furthermore, the labelling of a first element does not imply the presence of a second element and vice versa.

It may be appreciated that FIGS. 1-2C comprise some modules or operations which are illustrated with a solid line and some modules or operations which are illustrated with a dashed line. The modules or operations which are comprised in a solid line are modules or operations which are comprised in the broadest example embodiment. The modules or operations which are comprised in a dashed line are example embodiments which may be comprised in, or a part of, or are further modules or operations which may be taken in addition to the modules or operations of the solid line example embodiments. It should be appreciated that these operations need not be performed in order presented.

Furthermore, it should be appreciated that not all of the operations need to be performed. The example operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the example embodiments may be implemented at least in part by means of both hardware and software, and that several “means”, “units” or “devices” may be represented by the same item of hardware.

The various example methods, devices, and systems described herein are described in the general context of method steps processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

Examples of methods and products (blood analyser and system) according to the disclosure are set out in the following items:

Item 1. A blood analyser, the blood analyser comprising a memory, an interface, and one or more processors, the blood analyser being configured to:
obtain image data of a prepared blood sample;
select a first image associated with a first image plane of the prepared blood sample from the image data;
characterize the first image, wherein the characterization of the first image comprises to determine a first set of cell regions belonging to the first image plane; and
determine a first blood parameter based on the first set of cell regions.

Item 2. Blood analyser according to item 1, wherein the blood analyser is configured to select, from the image data, a first distal image associated with a first distal image plane on a distal side of the first image plane, and wherein the determination of the first set of cell regions is based on the first distal image.

Item 3. Blood analyser according to any of items 1-2, wherein the blood analyser is configured to select, from the image data, a first proximal image associated with a first proximal image plane on a proximal side of the first image plane, and wherein the determination of the first set of cell regions is based on the first proximal image.

Item 4. Blood analyser according to any of items 1-3, wherein the characterization of the first image comprises to determine a first initial candidate set of candidate cell regions in the first image.

Item 5. Blood analyser according to item 4, wherein to determine the first initial candidate set of candidate cell regions comprises to determine a first background image of the first image, and wherein the first initial candidate set of candidate cell regions is based on the first background image.

Item 6. Blood analyser according to item 5, wherein to determine the first initial candidate set of candidate cell regions comprises to determine a first contrast image based on the first background image and the first image, and wherein the first initial candidate set of candidate cell regions is based on the first contrast image.

Item 7. Blood analyser according to item 6, wherein to determine the first initial candidate set of candidate cell regions comprises to determine a first binary image based on the first contrast image, and wherein the first initial candidate set of candidate cell regions is based on the first binary image.

Item 8. Blood analyser according to item 7, wherein to determine the first initial candidate set of candidate cell regions comprises to identify connected regions in the first binary image, and wherein the first initial candidate set of candidate cell regions is based on the connected regions in the first binary image.

Item 9. Blood analyser according to item 8, wherein to determine the first initial candidate set of candidate cell regions comprises to determine whether each respective connected region satisfies an area criterion, and in accordance with the determination that the respective connected region satisfies the area criterion, to include the respective connected region satisfying the area criterion as a candidate cell region in the first initial candidate set of candidate cell regions.

Item 10. Blood analyser according to item 9, wherein the area criterion comprises a threshold cell region area in the range of 1 μm² to 25 μm².

Item 11. Blood analyser according to any of items 4-10, wherein the characterization of the first image comprises to determine whether each of the respective candidate cell regions of the first initial candidate set of candidate cell regions satisfies a first criterion, and in accordance with the determination that a respective candidate cell region of the first initial candidate set of candidate cell regions respectively satisfies the first criterion, to include the respective candidate cell region in a first candidate set of cell regions, and wherein the first set of cell regions is based on the first candidate set of candidate cell regions.

Item 12. Blood analyser according to item 11 as dependent on item 2 and item 3, wherein the first criterion is based on a first distal contrast parameter of the first distal image and a first proximal contrast parameter of the first proximal image, and wherein to determine whether each of the respective candidate cell regions of the first candidate set of cell regions satisfies a first criterion comprises to determine whether a first contrast parameter of the first contrast image is larger than the first distal contrast parameter and larger than the first proximal contrast parameter.

Item 13. Blood analyser according to any of items 11-12, wherein the characterization of the first image comprises to determine whether each of the respective cell regions of the first candidate set of candidate cell regions satisfies a second criterion, and in accordance with the determination that a respective cell region of the first candidate set of candidate cell regions respectively satisfies the second criterion, to include the respective cell region in a second candidate set of candidate cell regions, and wherein the first set of cell regions is based on the second candidate set of candidate cell regions.

Item 14. Blood analyser according to any of items 1-13, wherein the first image plane is associated with a first height in the prepared blood sample.

Item 15. Blood analyser according to any of items 1-14 as dependent on item 2, wherein the first distal image plane is associated with a first distal height in the prepared blood sample, the first distal height being different from the first height.

Item 16. Blood analyser according to any of items 1-15 as dependent on item 2 and 3, wherein a first distal distance between the first image plane and the first distal image plane, and a first proximal distance between the first image plane and the first proximal image plane are equal.

Item 17. Blood analyser according to any of items 1-16 as dependent on item 3, wherein the first proximal image plane is associated with a first proximal height in the prepared blood sample, the first proximal height being different from the first height.

Item 18. Blood analyser according to any of items 1-17, wherein the cell regions are indicative of one or more platelets.

Item 19. Blood analyser according to any of items 1-18, wherein the blood analyser is configured to:
select a second image associated with a second image plane of the prepared blood sample from the image data;
characterize the second image, wherein the characterization of the second image comprises to determine a second set of cell regions belonging to the second image plane; and
wherein to determine a first blood parameter is based on the second set of cell regions.

Item 20. Blood analyser according to item 19, wherein to determine a first blood parameter based on the first set of cell regions and the second set of cell regions comprises to determine a first number of cell regions in the first set of cell regions and a second number of cell regions in the second set of cell regions, and wherein the first blood parameter is based on the first number and the second number.

### LIST OF REFERENCES

1 user
2 system
4 output
6 transmit/obtain
10 blood analyser
10A memory
10B interface
10C processor
20 microscope
22 container/cuvette
24 central portion
100 method of analysing a prepared blood sample
S102 obtaining image data
S104 selecting image
S104A selecting first image
S104B selecting second image
S104C selecting third image
S104D selecting fourth image
S104E selecting fifth image
S106 characterizing image
S106A determining set of cell regions
S106B determining initial candidate set of cell regions
S110 determining a first candidate set of cell regions
S112 determining a contrast parameter of the image, determining a distal contrast parameter of the distal image, determining a proximal contrast parameter of the proximal image
S114 determining whether each of the respective candidate cell regions satisfies a first criterion
S116 including the respective candidate cell region in a first candidate set of cell regions
S118 determining a second candidate set of candidate cell regions
S120 initialising index
S122 determining whether each of the respective candidate cell regions of the first candidate set of candidate cell regions satisfies a second criterion
S124 including the respective candidate cell region in a second candidate set of cell regions
S126 determining whether enough images have been selected
S130 determining background image
S132 determining contrast image
S134 determining binary image
S136 identify connected regions
S137 discarding the respective connected region
S138 determining whether each respective connected region of the connected regions satisfies an area criterion
S139 including the respective connected region satisfying the area criterion as a candidate cell region in the initial candidate set of candidate cell regions
S140 determining a first blood parameter based on the set of cell regions, such as the first set of cell regions
S142 outputting the first blood parameter
A proceed
B reiterate, restart
C proceed
PIP_i proximal image plane
PIP_1 first proximal image plane
IP_i image plane
IP_1 first image plane
DIP_i distal image plane
DIP_1 first distal image plane
PH_i proximal height
PH_1 first proximal height
H_i height
H_1 first height
DH_i distal height
DH_1 first distal height
PD_i proximal distance
DD_i distal distance
PD_1 first proximal distance
DD_1 first distal distance
CE_1 first cell
Δz stepping incrementation
z z-axis
ID image data
I_i image
PI_i proximal image
DI_i distal image
SCR_i set of cell regions
CR_k cell regions
BP_1 first blood parameter
C_i number of cell regions
ICCR_i initial candidate set of candidate cell regions
CCR_f candidate cell regions of the initial set of candidate cell regions
CCR_fi candidate cell regions of the first set of candidate cell regions
BG_i background image
CI_i contrast image
BI binary image
COR_i connected regions
AC_m area criterion
FCCR_i first candidate set of candidate cell regions
SCCR_i second candidate set of candidate cell regions
FC_i first criterion
SC_i second criterion
PCP_i proximal contrast parameter
CP_i contrast parameter
DCP_i distal contrast parameter
DCI_i distal contrast image
PCI_i proximal contrast image
TC third criterion

1. A blood analyser, the blood analyser comprising a memory, an interface, and one or more processors, the blood analyser being configured to:
obtain image data of a prepared blood sample, the image data comprising data of a stack of images, where each image of the stack of images is associated with an image plane, wherein each image plane is associated with a different height along a z-axis of the prepared blood sample;
select a first image associated with a first image plane of the prepared blood sample from the image data;
characterize the first image, wherein the characterization of the first image comprises determining a first set of cell regions belonging to the first image plane, wherein the cell regions are indicative of one or more platelets; and
determine a first blood parameter based on the first set of cell regions.

2. The blood analyser according to claim 1, wherein the blood analyser is configured to select, from the image data, a first distal image associated with a first distal image plane on a distal side of the first image plane, and wherein the determining of the first set of cell regions is based on the first distal image.

3. The blood analyser according to claim 1, wherein the blood analyser is configured to select, from the image data, a first proximal image associated with a first proximal image plane on a proximal side of the first image plane, and wherein the determining of the first set of cell regions is based on the first proximal image.

4. The blood analyser according to claim 1, wherein the characterization of the first image comprises determining a first initial candidate set of candidate cell regions in the first image.

5. The blood analyser according to claim 4, wherein determining the first initial candidate set of candidate cell regions comprises determining a first background image of the first image, and wherein the first initial candidate set of candidate cell regions is based on the first background image.

6. The blood analyser according to claim 5, wherein determining the first initial candidate set of candidate cell regions comprises determining a first contrast image based on the first background image and the first image, and wherein the first initial candidate set of candidate cell regions is based on the first contrast image.

7. The blood analyser according to claim 6, wherein determining the first initial candidate set of candidate cell regions comprises determining a first binary image based on the first contrast image, and wherein the first initial candidate set of candidate cell regions is based on the first binary image.

8. The blood analyser according to claim 7, wherein determining the first initial candidate set of candidate cell regions comprises identifying connected regions in the first binary image, and wherein the first initial candidate set of candidate cell regions is based on the connected regions in the first binary image.

9. The blood analyser according to claim 8, wherein determining the first initial candidate set of candidate cell regions comprises determining whether each respective connected region satisfies an area criterion, and in accordance with the determination that the respective connected region satisfies the area criterion, including the respective connected region satisfying the area criterion as a candidate cell region in the first initial candidate set of candidate cell regions.

10. The blood analyser according to claim 9, wherein the area criterion comprises a threshold cell region area in the range of 1 μm² to 25 μm².

11. The blood analyser according to claim 4, wherein the characterization of the first image comprises determining whether each of the respective candidate cell regions of the first initial candidate set of candidate cell regions satisfies a first criterion, and in accordance with the determination that a respective candidate cell region of the first initial candidate set of candidate cell regions respectively satisfies the first criterion, including the respective candidate cell region in a first candidate set of cell regions, and wherein the first set of cell regions is based on the first candidate set of candidate cell regions.

12. (canceled)

13. (canceled)

14. The blood analyser according to claim 1, wherein the first image plane is associated with a first height in the prepared blood sample.

15. (canceled)

16. (canceled)

17. The blood analyser according to claim 1, wherein the blood analyser is configured to:
select a second image associated with a second image plane of the prepared blood sample from the image data; and
characterize the second image, wherein the characterization of the second image comprises determining a second set of cell regions belonging to the second image plane;
wherein determining a first blood parameter is based on the second set of cell regions.

18. The blood analyser according to claim 17, wherein determining a first blood parameter based on the first set of cell regions and the second set of cell regions comprises determining a first number of cell regions in the first set of cell regions and a second number of cell regions in the second set of cell regions, and wherein the first blood parameter is based on the first number and the second number.

19. The blood analyser according to claim 1, wherein a distance between two image planes of the image data is in the range of 1 μm to 10 μm.

20. A computer program product comprising a non-transitory computer readable medium, having thereon a computer program comprising program instructions, the computer program being loadable into a data processing unit and configured to cause execution of the operations according to claim 1 when the computer program is run by the data processing unit.

21. The blood analyser according to claim 1,
wherein the blood analyser is configured to select, from the image data, a first distal image associated with a first distal image plane on a distal side of the first image plane, and wherein the determining of the first set of cell regions is based on the first distal image;
wherein the blood analyser is configured to select, from the image data, a first proximal image associated with a first proximal image plane on a proximal side of the first image plane, and wherein the determining of the first set of cell regions is based on the first proximal image;
wherein the characterization of the first image comprises determining whether each of the respective candidate cell regions of the first initial candidate set of candidate cell regions satisfies a first criterion, and in accordance with the determination that a respective candidate cell region of the first initial candidate set of candidate cell regions respectively satisfies the first criterion, including the respective candidate cell region in a first candidate set of cell regions, and wherein the first set of cell regions is based on the first candidate set of candidate cell regions; and
wherein the first criterion is based on a first distal contrast parameter of the first distal image and a first proximal contrast parameter of the first proximal image, and wherein determining whether each of the respective candidate cell regions of the first candidate set of cell regions satisfies a first criterion comprises determining whether a first contrast parameter of the first contrast image is larger than the first distal contrast parameter and larger than the first proximal contrast parameter.

22. The blood analyser according to claim 21, wherein the characterization of the first image comprises determining whether each of the respective cell regions of the first candidate set of candidate cell regions satisfies a second criterion, and in accordance with the determination that a respective cell region of the first candidate set of candidate cell regions respectively satisfies the second criterion, including the respective cell region in a second candidate set of candidate cell regions, and wherein the first set of cell regions is based on the second candidate set of candidate cell regions.

23. The blood analyser according to claim 1, wherein the blood analyser is configured to select, from the image data, a first distal image associated with a first distal image plane on a distal side of the first image plane, and wherein the determining of the first set of cell regions is based on the first distal image;
wherein the blood analyser is configured to select, from the image data, a first proximal image associated with a first proximal image plane on a proximal side of the first image plane, and wherein the determining of the first set of cell regions is based on the first proximal image;
wherein the first image plane is associated with a first height in the prepared blood sample; and
wherein the first distal image plane is associated with a first distal height in the prepared blood sample, the first distal height being different from the first height, and wherein the first proximal image plane is associated with a first proximal height in the prepared blood sample, the first proximal height being different from the first height.

24. The blood analyser according to claim 1, wherein the blood analyser is configured to select, from the image data, a first distal image associated with a first distal image plane on a distal side of the first image plane, and wherein the determining of the first set of cell regions is based on the first distal image;
wherein the blood analyser is configured to select, from the image data, a first proximal image associated with a first proximal image plane on a proximal side of the first image plane, and wherein the determining of the first set of cell regions is based on the first proximal image; and
wherein a first distal distance between the first image plane and the first distal image plane, and a first proximal distance between the first image plane and the first proximal image plane are equal.
